(19) United States
(12) Patent Application Publication
(10) Pub. No.: US 2005/0130149 A1
(43) Pub. Date: Jun. 16, 2005

(54) Method for williams-beuren syndrome diagnosis

(76) Inventors: Lucy R Osborne, Toronto (CA); Stephen W Scherer, Toronto (CA)

Correspondence Address: MYERS BIGEL SIBLEY & SAJOVEC, PO BOX 37428, RALEIGH, NC, 27627, US

(21) Appl. No.: 10/493,715

(22) PCT Filed: Oct. 25, 2002
(86) PCT No.: PCT/CA02/01611

Related U.S. Application Data
(60) Provisional application No. 60/330,571, filed on Oct. 25, 2001.

(51) Int. Cl. C12Q 1/68; C12P 19/34

(52) U.S. Cl. 435/6; 435/91.2

(57) ABSTRACT

A novel method is provided for identification of WBS patients or carriers by detection of an inversion in the WBS region of chromosome 7.

### FIELD OF THE INVENTION

The present invention relates to novel genomic polymorphisms located on chromosome 7 and their use in methods for the detection of Williams-Beuren Syndrome or a carrier of Williams-Beuren Syndrome.

### BACKGROUND OF THE INVENTION

Throughout this application, various references are cited in parentheses to describe more fully the state of the art to which this invention pertains. The disclosures of these references are hereby incorporated by reference into the present disclosure.

The Williams-Beuren Syndrome (WBS), also known as Williams Syndrome, occurs in approximately 1 in 20,000 individuals worldwide. The phenotype includes congenital vascular and heart disease, dysmorphic facies, growth deficiency, infantile hypercalcemia, mental retardation, a unique cognitive profile and a characteristic personality (Greenberg, F. (1990); Pober et al., (1996)).

Presently, diagnosis of WBS includes testing for a hemizygous deletion at 7q11.23 by fluorescence in situ hybridization (FISH) using a probe encompassing the elastin gene (ELN) (Ewart et al., (1993)). In over 95% of WBS cases, there is observed a defined hemizygous 1.5 Mb deletion (FIG. 1) which includes the elastin locus (ELN). For the remaining individuals evidencing a WBS phenotype, there has been, up till now, no recognised chromosomal rearrangement. In atypical patients having a subset of the symptoms, other chromosome rearrangements, usually affecting 7q11.23 but also other parts of chromosome 7, have been described (Table 1) (Mari et al., (1995); Frangiskakis et al., (1996); Botta et al., (1999); von Dadelszen et al., (2000)). U.S. Pat. No. 5,560,282 describes a method for diagnosing WBS based on detecting the presence of only a single copy of the elastin gene.

Due to the sporadic nature of the microdeletions that occur, there is currently no screening method for testing extended family members who may be predisposed to have a WBS child.

Thus there remains a need for improved diagnostic methods for Williams-Beuren Syndrome and for carriers.

### SUMMARY OF THE INVENTION

The present invention discloses novel genomic polymorphisms comprising inversions within the WBS region (7q 11.23 region) of chromosome 7 which encompass multiple genes within this region of chromosome 7. These novel genomic polymorphisms can be used in methods for determining individual carriers of the inversions and thus the likelihood of having children with WBS. The methods of the present invention can also be used for the diagnosis of Williams-Beuren Syndrome. Prenatal screening methods are included within the scope of the invention and predictive tests are also provided to determine whether an individual is at risk of having a child with WBS.

With the identification of novel, inversions in the WBS region, nucleic acid probes can be used in a variety of hybridization assays to screen for and detect the presence of the inversions. Kits for such screening and diagnosis are also provided.

In accordance with an aspect of the present invention is a gene region comprising the 7q11.23 WBS region containing an inversion leading to a syndrome or disease profile in an individual.

In accordance with a further aspect of the invention is an inversion comprising a 1.5 Mb gene region of 7q11.23

In accordance with another aspect of the invention is a method for determining the presence or absence of an inversion within the WBS region of chromosome 7 in a subject, the method comprising the steps of: i) analyzing cells from an individual using a genomic hybridization technique; and

ii) detecting the presence or absence of an inversion in the WBS region.

In one embodiment, the genomic hybridization technique used is fluorescent in situ hybridization (FISH).

In accordance with another aspect of the invention is a method for determining whether a subject displaying Williams-Beuren Syndrome (WBS)-associated symptoms suffers from WBS, or an asymptomatic subject is a carrier of WBS, the method comprising:

obtaining a biological sample from the subject; and

conducting an assay on the sample to determine the presence or absence of at least one inversion in the WBS region of chromosome 7, the presence of at least one inversion being indicative that the subject displaying symptoms suffers from WBS or that the asymptomatic subject is a carrier of WBS.

In a preferred embodiment, the method comprises using three colour FISH analysis on lymphocytes which have been synchronized in interphase. Two probes are designed to hybridize with specific sequences within the WBS deletion region and a third probe hybridizes to a region which flanks the WBS region. The relative positioning of the probes determines the presence of an inversion.

According to another aspect of the invention is a method for determining the presence of a Williams-Beuren Syndrome (WBS)-associated inversion, the method comprising the steps of:

i) digesting DNA from an individual with a restriction enzyme to obtain restriction fragments;

ii) size fractionating said fragments; and

iii) determining the presence or absence of novel restriction fragments, indicating the presence of a WBS inversion mutation.

In a preferred embodiment, the DNA is digested with Not1 enzyme. It is well known to one skilled in the art that other restriction enzymes which provide a restriction map which differs from that seen in the normal can also be used in the above method.

According to a further aspect of the invention is a method for the detection of a WBS-associated inversion comprising the steps of:

i) subjecting DNA or RNA from a subject to amplification using oligonucleotide primers, wherein at least one primer is complementary to a nucleic acid sequence upstream of a breakpoint and at least one primer is complementary to a nucleic acid sequence downstream of said breakpoint and wherein said primers are complementary to opposite strands of nucleic acid;

ii) amplifying the genetic region between the primers using a polymerization chain reaction technique and obtaining an amplification product;

iii) determining the sequence of the amplification product; and

iv) comparing the sequence of the amplified region with the sequence from a control individual to determine the presence of an inversion.

In yet another aspect of the invention, there is provided a method for determining the risk of having a child with WBS or other syndromes associated with an inversion polymorphism in the WBS region. The method comprises screening family members for the presence or absence of an inversion polymorphism in the 7q11.23 gene region

In yet another aspect of the invention, there is provided a kit for determining the Williams-Beuren genotype of an individual, said kit comprising at least one internal probe labeled with a fluorophore, said internal probe being able to hybridize to a sequence within the WBS region of chromosome 7 and at least one external probe labeled with a different fluorophore, said external probe being able to hybridize to a region flanking said WBS region.

Other features and advantages of the present invention will become apparent from the following detailed description. It should be understood, however, that the detailed description and the specific examples, while indicating embodiments of the invention, are given by way of illustration only, since various changes and modifications within the spirit and scope of the invention will become apparent to those skilled in the art from said detailed description.

### DESCRIPTION OF THE DRAWINGS

The invention is described in more detail herein with reference to the accompanying drawings, in which:

FIG. 1 shows the WBS region at 7q11.23. The rearrangement breakpoints in translocation patient 11719 and inversion patient 15441, as determined by FISH, are shown, as well as the NotI sites for PFGE. The four probes used for interphase FISH as illustrated in FIG. 2, ie. RP11-815K3, CTA-208H19, RP5-1186P10 and CTB-139P11, are indicated by large shaded circles. The 18 probes used to fine-map the inversion breakpoints and to test for subtle chromosome rearrangements, are shown (left to right: RP11-421B22 (AC006334), RP5-845121 (AC004905), RP4-63505 (AC004845), HSC7E610, CTB-23115 (AC005049), CTA-208H19 (AC005074), CTA-315H11 (AC005089), cos16g10 (U87315, U87314, U87310 (STX1A exon sequences)), cos82c2 (exon trapping), cos34b3 (U63721), RP11-122H9 (AQ383842, AQ343610 (end sequences)), cos209c11 (exon trapping), RP11-267N24 (exon trapping), RP11-54H15 (AQ081580, AQ115183 (end sequences)), CTB-139P11 (AC004491), CTA-356E1 (AC005102), CTB-122E10 (AC005067), HSC7E139, RP5-1186P10 (AC005231) and RP11-815K3 (AC0067941)). (Accession number in parentheses are with respect to NCBI Nucleotide Database).

Genes are depicted as arrows where the transcriptional orientation (5′ to 3′) is known, and as blocks where it is not known. DNA sequence scaffolds from Celera (component 3 assembly) and the public genome project are shown. Repetitive gene sequences within the duplicons are identified by degree of shading in the legend below left and the duplicons themselves are presented as large vertical shaded boxes. The minimal regions within which the inversion breakpoints could be localized are depicted by diagonal hatched boxes.

FIG. 2 shows the order of FISH probes used along a normal chromosome 7 and, except for control sample, a chromosome with the inversion. Panel A: probes from centromere to telomere: CTA-208H19 (green), RP5-1186P10 (yellow) and CTB-139P11 (red). Panel B: probes from centromere to telomere: RP 11-815K3 (red), CTA-208H19 (green) and RP5-1186P10 (yellow).

FIG. 3 shows the use of multiple test probes to determine the translocation and extent of inversion in a WBS patient;

FIG. 4 shows polymorphic DNA marker analysis in 3 WBS families; P=proband, F=father, M=mother.

FIG. 5 shows the presence of a novel Not1-PFGE restriction fragment in individuals carrying a WBS region inversion by PFGE fractionation and blot-hybridization analysis using a GTF 21-specific probe. A representative result with a resolution of fragments in the 450 kb-1.6 Mb range is shown.

### DETAILED DESCRIPTION OF THE INVENTION

The present invention discloses previously undescribed inversions of a portion of chromosome 7 in a number of atypical WBS patients and in asymptomatic relatives of these patients. The first group of inversions detected involves an approximately 1.5 Mb region of 7q11.23. The patients either did not show all the typical symptoms of WBS or they did not exhibit the previously identified WBS deletion. The clinical features of the WBS families studied are outlined in Table 1.

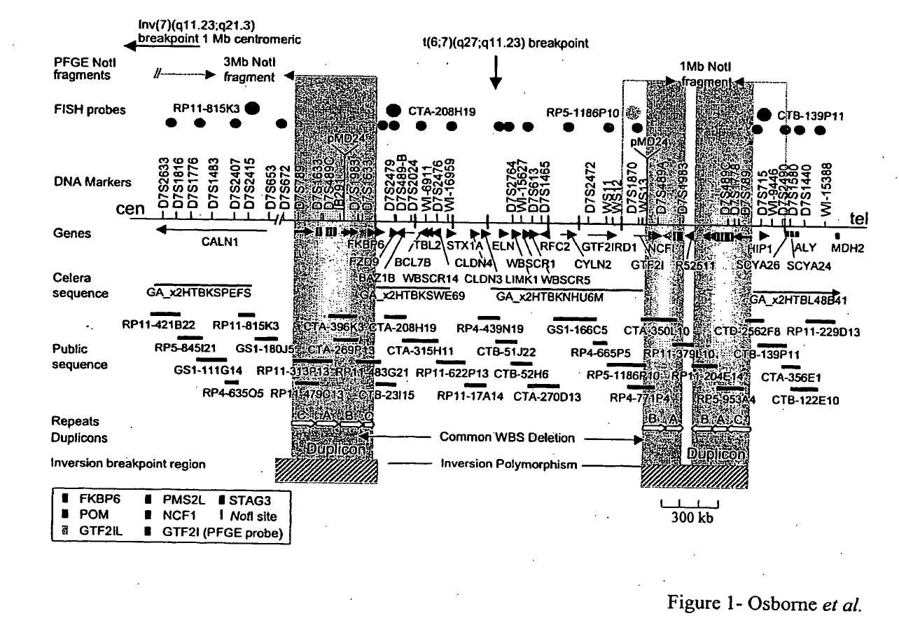

The intrachromosomal segmental DNA duplications flanking the WBS region (often called duplicons) were analysed and determined to be approximately 400 kb in size (FIG. 1). These segmental duplications are comprised of blocks of near identical DNA (>95% identity) occurring in the same and opposite orientations. They contain transcribed genes, conserved pseudogenes with near identical genomic structure, pseudogenes corresponding to their ancestral progenitors found at other sites on chromosome 7, and putative telomere-associated repeats. Misalignment and unequal cross-over of DNA sequences positioned in direct or inverted orientations within each of the larger duplicons could lead to a deletion as observed in WBS (or duplications which have not yet been observed), or to an inversion, respectively, of the intervening region. In view of these findings as well as observations made in other diseases, it was believed that an undetected genomic variation might exist at 7q11.23 contributing to the pathogenesis and/or mechanism underlying WBS.

To test for the presence of chromosomal inversion or duplication at 7q11.23, interphase FISH analysis was initially performed on 11 atypical WBS individuals and their parents (when available). The atypical individuals were divided into two groups, (i) 3 patients having an inversion or translocation on chromosome 7 and (ii) 8 patients with no detectable cytogenetic chromosomal rearrangement (Table 1).

An inversion of the 7q11.23 WBS region on the rearranged chromosome was identified in atypical individuals 11719 and 15441, who carried a balanced translocation and a paracentric inversion, respectively (Table 1). The inversion was also detected in an atypical WBS patient (12503) who, upon extensive FISH analysis, did not appear to have any other chromosomal anomaly. The father of individual 11719 and the mother of individual 15441, both phenotypically normal, also carried a hemizygous inversion of the WBS region. No rearrangement was found in the other atypical WBS patients as indicated in Table 1.

The unexpected observation of an inversion being present in parents of two unrelated atypical WBS individuals carrying more complex chromosome rearrangements, suggested that it would be beneficial to examine parents of typical WBS patients who carry the microdeletion. A diagnostic test of probability can be developed based on the rationale that, in a parent carrying an inversion on one chromosome, there may be difficulties in chromosome pairing at meiosis leading to the WBS deletion. Using the same interphase FISH assay, a heterozygous inversion was found in 4 of 12 (33%) families examined. These results are shown in Table 1.

In addition to the data shown in Table 1, this inversion was found in three further WBS patients in three separate new families.

Breakpoints for the observed inversions occurred between the position of probes RP11-815K3 and CTA-208H19 on the centromeric side and between probes RP5-1186P10 and RP11-229D13 on the telomeric side.

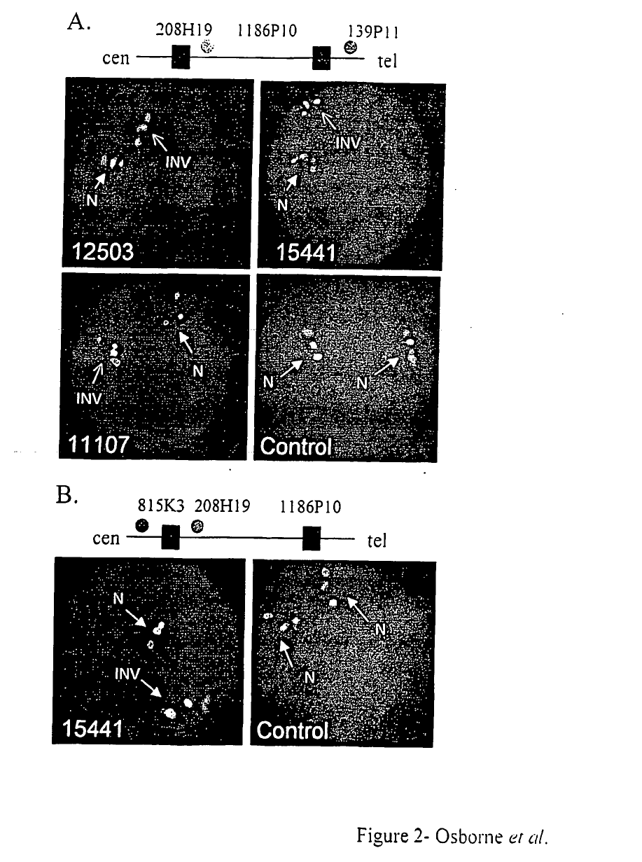

As discussed above, the presence of the inversion was detected by FISH analysis. Combinations of probes in three-color FISH experiments (with two probes from within the common deletion and one from the outside) were used to determine the orientation of the 1.5 Mb WBS region relative to flanking DNA as described in the following Examples. Changes in copy number due to duplications or smaller deletions can also be detected and the boundaries of chromosome rearrangement can also be defined. FIG. 2 shows in diagrammatic form the order of the three probes. In 2A, the FISH probes used comprise two “internal” probes (208H19/green and 1186P10/yellow) which hybridize to regions within the common deletion region and a third probe (139P11/red) which hybridizes to a region which is telomeric to the common deletion region. On a normal chromosome (N),the probes appear in the expected order, i.e. green-yellow-red. On an inverted chromosome (INV) the green signal appears between the red and yellow. In 2B, the same two “internal” probes were used in combination with a probe (815K3/red) which hybridizes to a region which is centromeric to the common deletion region. In this case, the expected order for a normal chromosome (N) is red-green-yellow. On an inverted chromosome, the green yellow signal appears between the red and green.

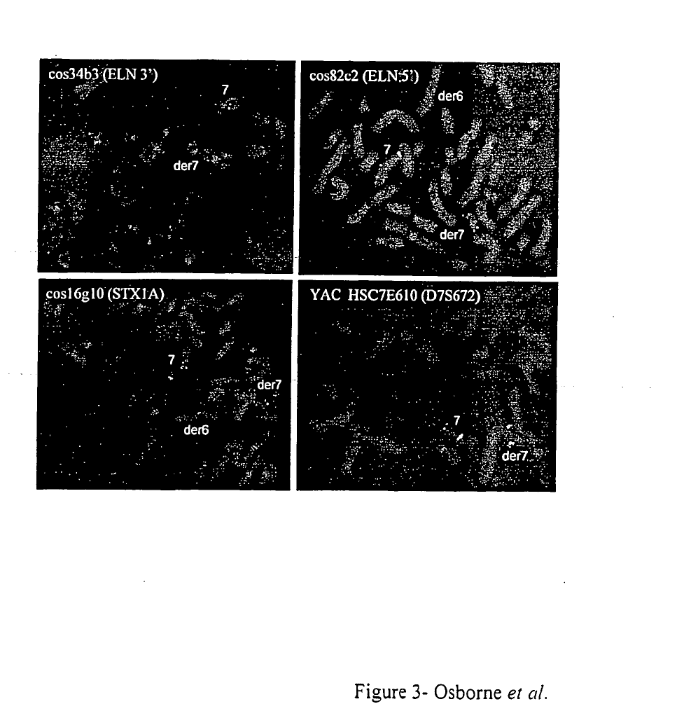

Using combinations of FISH probes to examine interphase and metaphase chromosome preparations, it was consistently demonstrated that the inversion breakpoints in each individual studied occurred within the duplicon region. For example, by examining metaphase chromosomes of a patient, the proximal and distal parts of the WBS region could be studied in isolation since they resided on different derivative chromosomes. The results of one such analysis is shown in FIG. 3. Atypical WBS patient 11719 with a balanced translocation was identified to carry the WBS inversion. The father (11976) of this patient also carried the inversion but not the translocation. A control probe on chromosome 7p22 (RP11-13N3 containing LFNG; red) was used to identify the derivative chromosome 7. Multiple test probes (green) were used to determine the site of the translocation and extent of the inversion in patient 11719. Based on the presence or absence of signals on the derivative chromosomes, the translocation breakpoint could be mapped to the immediate 5′ end of the ELN gene (see FIG. 1 for location; note that no deletions in the region were detectable). It was observed that probes cos34b3 and cos82c2 had the same pattern of hybridization to the translocation chromosomes as HSC7E610 (ie. hybridizing to the derivative chromosome 7), while cos16g10 hybridized to the derivative chromosome 6. This suggests a probe order of HSC7E610 (D7S672)-[cos34b3 (3′-ELN)-cos82c2 (5′-ELN)]-cos16g10 (STX1A). However, on a normal chromosome the known order of probes is: 7cen-HSC7E610 (D7S672)-duplicon-cos16g10 (STX1A)-cos82c2 (5′-ELN)-cos34b3 (3′-ELN)-duplicon-7qter (FIG. 1). This indicates the presence of the WBS inversion in patient 11719. The combined results of testing 20 probes using the same strategy indicated that the inversion breakpoints in this patient occurred within the duplicons. Probes mapping outside the WBS region were always located on the expected derivative translocation chromosome. However, probes residing between either duplicon and the translocation breakpoint, which was mapped to the 5′ end of ELN as shown in FIG. 1, always hybridized to the derivative chromosome. This is opposite to what would be expected when compared to normal chromosomes.

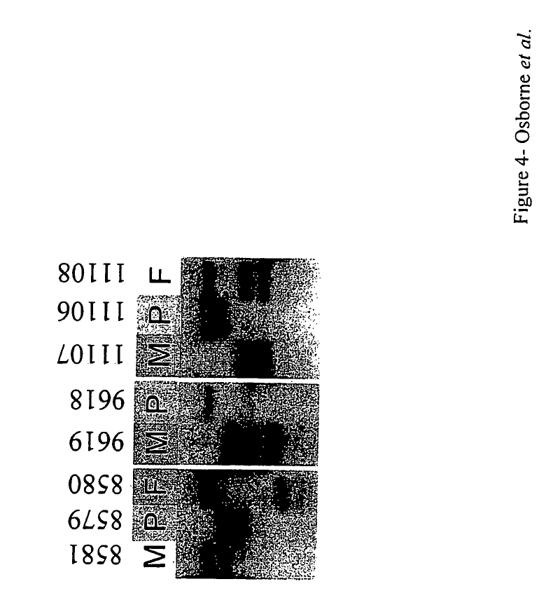

FIG. 4 illustrates that, in all cases, the inversion was present only in the parental genome (3 maternal, 1 paternal) transmitting the chromosome that had presumably undergone unequal recombination, as determined by polymorphic marker analysis. The inversion was not found in the non-transmitting WBS parents. It was also not found in 7 of the 8 atypical WBS individuals with no cytogenetically visible chromosome rearrangement (Table 1), or in the 26 unrelated non-WBS control individuals. Chromosomal duplication was not observed in any of the cases studied.

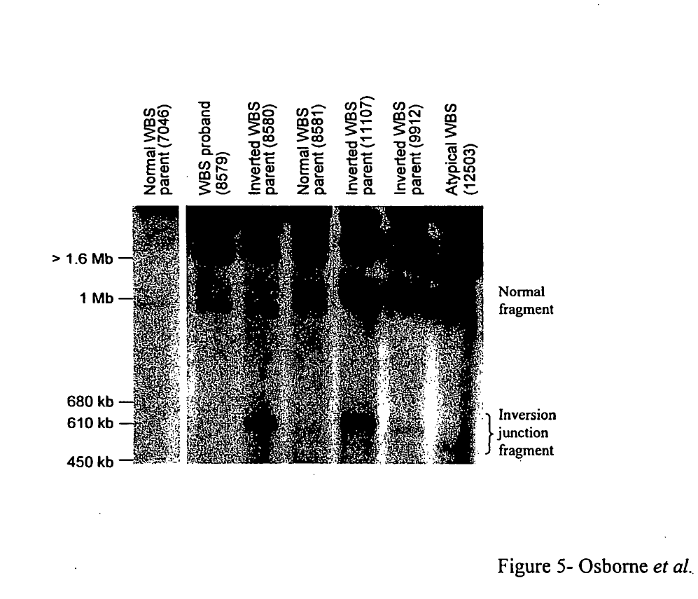

The presence of the inversion may be detected by any suitable method known to those of skill in the art, including, for example, genomic hybridization techniques, PCR analysis and restriction fragment analysis. FIG. 5 illustrates the presence of a new Not1-PFGE restriction fragment in individuals carrying the WBS region inversion that is not present in normal individuals. The novel fragment ranging from 500-600 kb in size, appeared only in individuals shown by FISH to carry the inversion. Generation of a new Not1 fragment in this size range is consistent with the expected structural changes in the chromosome arising from an inversion occurring via recombination between the flanking duplicons (FIG. 1). Variation in size of the Not1 junction fragments could be due to the breakpoints occurring within different segments of inverted blocks of repeat between the duplicons (Blocks A, B, C, of FIG. 1). As shown in FIG. 5, the phenotypically normal inversion breakpoint carriers studied by PFGE (8580, 11107, 9912) all had similar sized NotI junction fragments (600 kb), while an atypical WBS individual (12503) carried a smaller fragment approximately 500 kb in size. A subtle rearrangement accompanying the inversion or polymorphism may contribute to this difference.

The presence of the described inversion may also be detected by PCR analysis, by amplification of a portion of chromosome 7 spanning an inversion breakpoint, using suitable primers and sequencing of the amplified portion. The presence of an abnormal junction sequence, resulting from inclusion of an inverted sequence within the amplified portion, indicates the presence of the inversion.

The present invention provides the unexpected demonstration that the WBS region can undergo two relatively large genomic rearrangements, deletion and inversion, both apparently mediated by the repetitive units flanking the interval. The presence of the above-described inversion in parents of WBS children suggests that this genomic variant may lead to disturbances in meiosis predisposing to chromosome rearrangements in future generations. In WBS, since the number of reported families with more than one affected individual is small, this would be a rare event. Most inversion carriers do not exhibit any obvious phenotypic features although in at least two individuals (12503 and 15441) the inversion seems to be associated with many WBS symptoms (Table 1). The data indicates that the inversion breakpoints in these 2 individuals reside within the duplicons, just as in each of the other phenotypically normal WBS inversion cases studied. It appears from PFGE experiments that the site of the breakpoint(s), or extent of rearrangement, in individual 12503 could be different. It may be that breakpoint(s) in 12503 and 15441 interrupt or affect the expression of functional gene(s) located within or near the duplicon or there may be another mutation or rearrangement. GTF2I is one such gene that could be affected since it is located partially inside the telomeric duplicon. Moreover, GTF2I was found deleted in atypical WBS patients carrying sub-WBS deletions having a similar phenotype to individuals 12503 and 15441.

The majority (˜67%) of WBS interstitial deletions have been shown to be due to unbalanced recombination during meiosis (interchromosomal rearrangement) while less (˜33%) appear to arise due to intrachromosomal recombination³. To determine if the inversion polymorphism is associated with one or both events and to provide better estimates of recurrence risk to siblings, the analysis can be expanded to include grandparents and siblings. The finding of the present invention that ˜30% of transmitting chromosomes in WBS families carry a genomic inversion provides new insight into the mechanism underlying the disease. The FISH and PFGE tests described for detection of the inversion are valuable in assisting in clinical diagnosis of WBS and for screening methods involved in family planning.

Further studies were carried out on patients or family members who, from the studies described above, did not appear to have either a deletion or the inversion described above. A new inversion at a different location in the WBS region of chromosome 7 (second inversion) was detected in one of these patients and in a parent of that patient. In the second inversion, breakpoints occurred between the position of probes CTA-208H19 and RP5-1186P10 on the centromeric side and between probes CTB-139P11 and RP11-275G11 on the telomeric side.

Four WBS patients in different families have been identified who have both the first described and second inversions and in some instances a parent was also shown to have both inversions.

The methods of the invention may be used for the diagnosis of patients presenting with classical WBS symptoms but showing no deletion of chromosome 7 or of patients presenting with some WBS-associated symptoms but lacking the full array of classical symptoms. WBS-associated symptoms include congenital vascular disease, congenital heart disease, dysmorphic facies, growth deficiency, infantile hypercalcemia and mental retardation.

The methods may also be used to screen members of a family with a history of WBS, as a basis for genetic counselling, as the finding of a WBS-associated inversion will indicate a possibility of bearing a child with WBS.

The methods may also be used for in utero testing to determine if a fetus carries a WBS-associated inversion.

### EXAMPLES

The examples are described for the purposes of illustration and are not intended to limit the scope of the invention.

Methods of molecular biology, molecular hybridization, protein and peptide biochemistry, histology and genetics referred to but not explicitly described in this disclosure and examples are reported in the scientific literature and are well known to those skilled in the art.

### Example 1

Patient Samples and Controls.

WBS probands were identified through the University of Arizona Genetics Program (15441), Virginia Commonwealth University (12503), Yale University School of Medicine (4 atypical individuals), McMaster University Medical Centre (1 atypical individual), and The Hospital for Sick Children, Toronto (remaining atypical and all classic WBS individuals). WBS was diagnosed using recognized diagnostic criteria. Local guidelines for human subject experimentation were followed. Proband 11719 was a neonate with severe supravalvular aortic stenosis and hydrops fetalis (von Dadelszen et al., (2000)), who died shortly after birth. Proband 12503 is an 18 year old female with WBS-like facial features, developmental delay, hypersensitivity to sound, malocclusion, strabismus, joint tightness, and WBS-like behaviour. Proband 15441 is a 16 year old female with ectrodactyly of the feet, WBS-like facial features, strabismus, musculoskeletal abnormalities, mild developmental delay, recurrent otitis media resulting in hearing loss, hyperactivity and WBS-like behaviour. In addition to the WBS inversion, she was also shown to have a larger inversion on chromosome 7 with breakpoints at 7q11.2 (over 1 Mb proximal to the WBS deletion, near D7S726) and at 7q21.3 (within the SHFM1 critical region). As controls for the WBS families (which were of different ethnic backgrounds), 26 randomly selected non-WBS cell lines and blood samples from ethnically diverse sources were used (52 chromosomes).

Mapping, DNA Sequence Analysis, DNA Markers, and Microsatellite Experiments.

An integrated genetic, physical, DNA sequence, and gene map spanning the WBS region was assembled for this study using new mapping data, published data (Osborne et at., (1997); Peoples et al., (2000); Valero et al., (2000)) and by annotating all available DNA sequence (FIG. 1)

The intrachromosomal segmental DNA duplications flanking the WBS region (often called duplicons) were analyzed and determined to be approximately 400 kb in size (FIG. 1). These segmental duplications are comprised of blocks of near identical DNA (>95% identity) occurring in the same and opposite orientations. The duplicons consist of actively transcribed genes (FKBP6, GTF2I, GTF2IL and NCF1), highly conserved pseudogenes with near identical genomic structure (GTF2IP1, GTF2IP2, NCF1P1, NCF1P2, GTF2ILP1, FKBP6P1, FKBP6P2), pseudogenes corresponding to ancestral progenitors found at other sites on chromosome 7 (PMS2-like genes, three STAG3 pseudogenes, and POM pseudogenes) (Perez-Jurado et al., (1998); Osborne et al. (1997); Pezzi et al., (2000); Görlach et al., (1997); Chute et al., (1997) and Meng et al. (1998)).

Percent Identity Plot (PIP) analysis was performed on the completed sequence of clones CTA-350L10, CTA-269P13, RP11-313P13 and RP5-953A4 using the automated analysis server PipMaker (Schwartz et al., (2000). Additional genetic markers for the studies were generated, including WS13 (D7S3197)(FIG. 4). Polymorphic marker analysis was carried out by gel electrophoresis and hybridization of products PCR-amplified from peripheral blood lymphocyte genomic DNA.

Fluorescence in situ Hybridization.

The protocols for FISH analysis were based on previously described techniques (Heng et al., (1992 & 1993)). Briefly, for metaphase analysis, lymphocytes were cultured for 68-72 hr, synchronized with BrdU (0.18 μg/ml, Sigma), washed and then re-cultured for 6 hr in α-MEM with thymidine (2.5 μg/ml, Sigma). Cells were harvested and slides were prepared by hypotonic treatment, fixed, and air-dried. For interphase analysis, slides from cell lines or peripheral blood were dried at room temperature for 3 days. Before hybridization, all slides were denatured in 70% formamide/2×SSC for 30 seconds at 70° C., and dehydrated with ethanol. Genomic DNA was isolated from clones using standard methods (e.g. Heng et al. 1992) and labeled with either biotin (green) or digoxigenin (red) to generate 500 bp fragments. For the dual color signal (yellow), DNA was labeled separately with biotin and digoxigenin and then mixed. Standard labeling techniques were used (Heng et al. 1992, 1993).

Probes were denatured for 5 min at 75° C. and hybridized to slides overnight (37° C. in 50% formamide with CotI DNA). The slides were washed, a detection solution was added and following DAPI staining, the slides were examined under a fluorescence microscope. Any standard labeled antibody solution can be used as the detection solution to detect the tag (e.g. biotin) on the probe. The detection efficiency on metaphase chromosomes was 90-99%, enabling assessment of the position of the hybridization signal relative to the rearrangement breakpoint. The WBS patients, family members, and controls were randomized so there was no bias of interpretation of results. Approximately 100 mitotic figures were examined for each probe tested. For assessment of interphase cells, only those chromosomes where all three probes could be visualized in close alignment with each other were scored (at least 25 chromosomes were scored for each individual).

Pulsed-field Gel Electrophoresis

Patient lymphoblast cell lines were embedded in low melting-point agarose at a concentration of ˜10⁷ cells/ml. High molecular weight DNA was prepared by incubation with sodium sarkosyl and Proteinase K. The DNA blocks were digested with NotI restriction endonuclease (New England Biolabs) and size fractionated through 1% agarose using a CHEF-II apparatus. Multiple gels under different conditions were run. Undigested Saccharomyces cerevisiae chromosomes were used as a size marker. The gels were transferred to nylon membranes and hybridized in Ambion hybridization buffer at 50° C., followed by washing at 55° C.

Accession Numbers

The DNA sequences generated in the present invention were submitted to GenBank. The GenBank accession numbers are G68164 (WS10), G68161 (WS11), G68162 (WS12), G68163 (WS13), AF020782 (pMD24), AZ757825/AZ757826 (cos24g11).

Detection and Characterisation of WBS-associated Inversion

Interphase FISH analysis of 11 atypical WBS individuals and available parents was performed as described above. Table 1 indicates the karyotype and phenotype of the subjects in the study.

Two different clone sets were used for FISH, both with two probes, 208419 and 1186P10, from within the common WBS deletion interval, but with the third either telomeric (A) or centromeric (B) to the region. The order of probes along a normal chromosome 7 are depicted in FIG. 2.

FIG. 2A:

The probes, from centromere to telomere, are CTA-208H19 (green), RP5-1186P10 (yellow) and CTB-139P11 (red)(FIG. 1). On a normal chromosome, the signals appear in the expected order. On an inverted chromosome, the green signal appears between the red and yellow, indicating that an inversion of the region has occurred.

FIG. 2B:

The probes are, from centromere to telomere, RP11-815K3 (red), CTA-208H19 (green), and RP5-1186P10 (yellow). On an inverted chromosome, the yellow signal appears between the red and green, indicating an inversion of the region. From the combined data, as well as using additional probes, it was demonstrated that the inversion breakpoints resided within the duplicon region (FIG. 1).

An inversion of the 7q11.23 WBS region on the rearranged chromosome was identified in atypical individuals 11719 and 15441, who carried both a balanced translocation and a paracentric inversion (Table 1). A control probe on chromosome 7p22 (RP11-13N3 containing LFNG, red) was used to identify the derivative chromosome 7. Multiple test probes (green) were used to determine the site of the translocation and extent of the inversion in patient 11719. Based on the presence or absence of signals on the derivative chromosomes, the translocation breakpoint was mapped to the immediate 5′ end of the ELN gene (see FIG. 1 for location; note that no deletions in the region were detectable).

It was observed that probes cos34b3 and cos82c2 had the same pattern of hybridization to the translocation chromosomes as HSC7E610 (ie. hybridizing to the derivative chromosome 7), while cos16g10 hybridized to the derivative chromosome 6. This suggests a probe order of HSC7E610 (D7S672)-[cos34b3 (3′-ELN)-cos82c2 (5′-ELN)]-cos16g10 (STX1A). However, on a normal chromosome the known order of probes is: 7cen-HSC7E610 (D7S672)-duplicon-cos16g10 (STX1A)-cos82c2 (5′-ELN)-cos34b3 (3′-ELN)-duplicon-7qter (FIG. 1), indicating the presence of the WBS inversion in patient 11719. The combined results of testing 20 probes using the same strategy, indicated that the inversion breakpoints in this patient occurred within the duplicons.

The inversion was also detected in an atypical WBS patient (12503), who upon extensive FISH analysis, did not appear to have any other chromosomal anomaly.

The father of individual 11719 and the mother of individual 15441, both phenotypically normal, also carried a hemizygous inversion of the WBS region. No rearrangement was found in the eight other atypical WBS patients, as indicated in Table 1.

### Example 2

Using the same interphase FISH assay, twelve parents of WBS probands with the common WBS deletion were examined. As shown in Table 2, a heterozygous inversion was found in four of the twelve families examined (33%).

### Example 3

Polymorphic DNA marker analysis was carried out using standard techniques in several WBS families. Polymorphic marker analysis throughout the WBS deletion region at 7q11.23 and the rest of chromosome 7 identified the microdeletion-containing chromosome in WBS probands to be inherited from the parent carrying the inversion. Representative results for three families with WS13 (D7S3197), a polymorphic (TAGA)ⁿ repeat marker that resides within the unique 5′ end of GTF2I and, therefore, within the WBS microdeletion, are shown in FIG. 4. The proband is designated (P), the father (F), and the mother (M). Proband 8579 exhibits loss of the paternal allele (8580), while proband 9618 and 11106 exhibit loss of the maternal allele (9619 and 11107). In each case, these are the parents that carry the inversion chromosome (see Table 1).

### Example 4

NotI-digested genomic DNA from WBS families was fractionated by PFGE and examined by blot-hybridization analysis using a GTF2I-specific probe (corresponding to the 3′ UTR of GTF2I; nucleotides 2134-2638 of GenBank NM_{—}032999). A representative result with resolution of fragments in the 450 kb to 1.6 Mb size range is shown. A novel NotI junction fragment was found in only those individuals (eg. 8580, 11107, 9912, 12503) shown by FISH to carry the WBS inversion. In normal individuals, the GTF2I probe should detect NotI fragments of 3 Mb and 1 Mb in size on the centromeric and telomeric side of the WBS region, respectively (GTF2I is present at each end of the WBS region and therefore hybridizes to two NotI fragments as seen in FIG. 1. The present results (lane 2; WBS proband 8579) and those previously described (Peoples et al., 2000), show that the 1.5 Mb microdeletion observed in WBS patients led to the formation of a 4 Mb junction fragment, in addition to the 3 Mb and 1 Mb NotI fragments present on non-deleted (normal) chromosomes. The 3 Mb and 4 Mb NotI fragments remain in the compression zone on this gel while the 1 Mb band is visible (lane 2). In carriers of the WBS inversion, the NotI junction fragment is in the 500-600 kb size range. This fragment size is consistent with what would be predicted if the inversion breakpoints occurred within the duplicons, as is known to be the case based on our FISH results (FIG. 1). Such an event would lead to a reduction in size of the 1 Mb NotI-fragment on the rearranged chromosome, as was observed when probing with GTF2I (the identity of the 500-600 kb new fragment was also confirmed by hybridization with a HIP1-gene probe). The extent in reduction of size of the 1 Mb NotI fragment would depend on the site of the inversion breakpoint(s) within the duplicon. In WBS parent 8581 (lane 4), who does not carry the inversion, a 1.1 Mb NotI fragment was observed in addition to the normal 1 Mb fragment. This may be due to size polymorphism within the WBS region occurring on one chromosome.

Although preferred embodiments of the invention have been described herein in detail, it will be understood by those skilled in the art that variations may be made thereto without departing from the spirit of the invention.

TABLE 1
Clinical features of families with WBS having the inversion polymorphism

| | | | |
|---|---|---|---|
| Individual/relationship | Karyotype | Phenotype | FISH |

(i) Individuals with atypical WBS with chromosomal rearrangement

| | | | |
|---|---|---|---|
| 11719 (proband) | 46XX t(7;7)(q27;q1111.23) | severe supravalvular aortic stenosis, | INV |
| | | hydrops fetalis; died shortly after birth | |
| 11976 (father of 11719) | 46XY | no clinical phenotype | INV |
| 15441 (proband) | 46XX inv(7)(q11.23;q21.3)^{b} | ectrodactyly, WBS facies, developmental delay, | INV |
| | | strabismus, WBS-like behaviour profile, I ordosis, chronic | |
| | | otitis media, normal growth, inattention | |
| 16582 (mother of 15441) | 46XX | no clinical phenotype | INV |
| 11532 (proband) | 46XY | WBS facies in childhood, | not INV |
| | t(6;7)(p10;p10) | developmental delay | |

(ii) individuals with atypical WBS without cytogenic rearrangement

| | | | |
|---|---|---|---|
| 12503 (proband) | 46XX | WBS facies, malocclusion, strabismus, joint tightness, | INV |
| | | hypersensitivity to sound, WBS-like behavior profile, | |
| | | developmental delay | |
| 16180 (mother of 12503) | 46XX | no clinical phenotype | not INV |
| 16179 (father of 12503) | 46XY | no clinical phenotype | not INV |
| plus seven other probands | four were 46XX | various subset of WBS symptoms | all were not INV |
| | three were 46XY | | |

(iii) transmitting parents of a WBS proband with the common deletion

| | | | |
|---|---|---|---|
| 11107 (mother) | 46XX | no clinical phenotype | INV |
| 8580 (father) | 46XY | no clinical phenotype | INV |
| 9619 (mother) | 46XX | no clinical phenotype | INV |
| 9912 (mother)) | 46XX | no clinical phenotype | INV |
| plus eight other | seven were 46XX | no clinical phenotype | all were not INV |
| transmitting parents | one was 46XY | | |

^{a}INV, WBS region inverted; not INV, WBS region not inverted.
^{b}The site of the 7q11.23 inversion breakpoint is indicated in FIG. 1. The 7q21.3 breakpoint is encompassed b cos24g11, located in the plit-hand-foot (SHFM1) critical region³⁰
^{c}P = 0.0038

TABLE 2
Transmitting parents of a WBS proband with the common deletion

| | | | |
|---|---|---|---|
| 11107 (mother) | 46 XX | No clinical phenotype | INV |
| 8580 (father) | 46 XY | No clinical phenotype | INV |
| 9619 (mother) | 46 XX | No clinical phenotype | INV |
| 9912 (mother) | 46 XX | No clinical phenotype | INV |
| Plus 8 other | Seven were 46 XX | No clinical phenotype | All |
| transmitting | One was 46 XY | | were |
| parents | | | Not |
| | | | INV |

### References

Baumer A. et al. High level of unequal meiotic crossovers at the origin of the 22q11.2 and 7q11.23 deletions. Hum. Mol. Genet. 7, 887-94 (1998).
Botta, A. et al. Detection of an atypical 7q11.23 deletion in Williams syndrome patients which does not include the STX1A and FZD3 genes. J. Med. Genet. 36, 478-480 (1999).
Chute, I., Le, Y., Ashley, T. & Dobson, M. J. The telomere-associated DNA from human chromosome 20p contains a pseudotelomere structure and shares sequences with the subtelomeric regions of 4q and 18p. Genomics 46, 51-60 (1997).
Crackower, M. A. et al. Characterization of the split hand/split foot malformation locus SHFM1 at 7q21.3-q22.1 and analysis of a candidate gene for its expression during limb development. Hum. Mol. Genet. 5, 571-579 (1996).
Ewart, A. K. et al. Hemizygosity at the elastin locus in a developmental disorder, Williams syndrome. Nat. Genet. 5, 11-6 (1993).
Frangiskakis, J. M. et al. LIM-kinase1 hemizygosity implicated in impaired visuospatial constructive cognition. Cell 86, 59-69 (1996).
Giglio S. et al. Olfactory receptor-gene clusters, genomic-inversion polymorphisms, and common chromosome rearrangements. Am. J. Hum. Genet. 68, 874-83 (2001).
Greenberg, F. Williams syndrome professional symposium. Am. J. Med. Genet. Suppl. 6, 85-88 (1990).
Görlach, A. et al. A p47-phox pseudogene carries the most common mutation causing p47-phox-deficient chromic granulomatous disease. J. Clin. Invest. 100, 1907-1918 (1997).
Heng, H. H., Squire, J. & Tsui, L. C. High-resolution mapping of mammalian genes by in situ hybridization to free chromatin. Proc. Natl. Acad. Sci. U S A. 89, 9509-13 (1992).
Heng, H. H. & Tsui, L. C. Modes of DAPI banding and simultaneous in situ hybridization. Chromosoma. 102, 325-32 (1993).
Ji, Y., Eichler, E. E., Schwartz, S. & Nicholls, R. D. Structure of chromosomal duplicons and their role in mediating human genomic disorders. Genome Res. 10, 597-610 (2000).
Jobling, M. A. et al. A selective difference between human Y-chromosomal DNA haplotypes. Curr. Biol. 8, 1391-4 (1998).
Kara-Mostefa, A. et al. Recurrent Williams-Beuren syndrome in a sibship suggestive of maternal germlne mosaicism. Am. J. Hum. Genet. 64, 1475-1478 (1999).
Lakich, D., Kazazian, H. H. Jr., Antonarakis, S. E. & Gitschier, J. Inversions disrupting the factor VIII gene are a common cause of severe haemophilia A. Nat. Genet. 5, 236-41 (1993).
Lowery, M. C. et al. Strong correlation of elastin deletions, detected by FISH, with Williams syndrome: evaluation of 235 patients. Am. J. Hum. Genet. 57, 49-53 (1995).
Mari, A. et al. Analysis of the elastin gene in 60 patients with clinical diagnosis of Williams syndrome. Hum. Genet. 96, 444-8 (1995).
Meng, X., Lu, X., Morris, C. A. & Keating, M. T. A novel human gene FKBP6 is deleted in Williams syndrome. Genomics 52, 130-137 (1998).
Nickerson, E., Greenberg, F., Keating, M. T., McCaskill, C. & Shaffer, L. G. Deletions of the elastin gene at 7q11.23 occur in approximately 90% of patients with Williams syndrome. Am. J. Hum. Genet 56, 1156-61 (1995).
Osborne, L. R. Williams-Beuren syndrome—unraveling the mysteries of a microdeletion disorder. Mol. Genet. Metab. 67, 1-10 (1999).
Osborne, L. R. et al. PMS2-related genes flank the rearrangement breakpoints associated with Williams syndrome and other disease on human chromosome 7. Genomics 45, 402-406 (1997).
Peoples, R. et al. A physical map, including a BAC/PAC clone contig, of the Williams-Beuren syndrome—deletion region at 7q11.23. Am. J. Hum. Genet. 66, 47-68 (2000).
Perez-Jurado, L. A. et al. A duplicated gene in the breakpoint regions of the 7q11.23 Williams-Beuren syndrome deletion encodes the initiator binding protein TFII-I and BAP-135, a phosphorylation target of BTK. Hum. Mol. Genet. 7, 325-334 (1998).
Pezzi, N. et al. STAG3, a novel gene encoding a protein involved in meiotic chromosome pairing and location of STAG3-related genes flanking the Williams-Beuren syndrome deletion. FASEB J. 14, 581-92 (2000).
Pober, B. R. & Dykens, E. M. Williams syndrome: An overview of medical, cognitive, and behavioural features. Child and Adolescent Psychiatric Clinics of North America 5:929-943 (1996).
Schwartz, S. et al. PipMaker—a web server for aligning two genomic DNA sequences. Genome Res. 10, 577-586 (2000).
Small, K., Iber, J. & Warren, S. T. Emerin deletion reveals a common X-chromosome inversion mediated by inverted repeats. Nat. Genet. 16, 96-9 (1997).
Tassabehji, M. et al. Williams syndrome: use of chromosomal microdeletions as a tool to dissect cognitive and physical phenotypes. Am. J. Hum. Genet. 64,118-25 (1999).
Valero M. C., de Luis O., Cruces J., Perez Jurado L. A. Fine-scale comparative mapping of the human 7q11.23 region and the orthologous region on mouse chromosome 5G: the low-copy repeats that flank the Williams-Beuren syndrome deletion arose at breakpoint sites of an evolutionary inversion(s). Genomics 69, 1-13 (2000).
von Dadelszen, P. et al. De novo 46,XX,t(6;7)(q27;q11;23) associated with severe cardiovascular manifestations characteristic of supravalvular aortic stenosis and Williams syndrome. Am. J. Med. Genet. 90, 270-5 (2000).

1. A method for determining whether a subject displaying Williams-Beuren Syndrome (WBS)-associated symptoms suffers from WBS, or an asymptomatic subject is a carrier of WBS, the method comprising:
obtaining a biological sample from the subject; and
conducting an assay on the sample to determine the presence or absence of at least one inversion in the WBS region of chromosome 7, the presence of at least one inversion being indicative that the subject displaying symptoms suffers from WBS or that the asymptomatic subject is a carrier of WBS.

2. The method of claim 1 wherein the at least one inversion comprises a centromeric breakpoint between the binding sites of probes RP11-815K3 and CTA-208H19 on chromosome 7 and a telomeric breakpoint between the binding sites of probes RP5-1186P10 and RP11-229D13 on chromosome 7.

3. The method of claim 1 wherein the at least one inversion comprises a centromeric breakpoint between the binding sites of probes CTA-208H19 and RP-1186P10 on chromosome 7 and a telomeric breakpoint between the binding sites of probes CTB-139P11 and RP11-275g11 on chromosome 7.

4. The method of claim 1 wherein two inversions are detected in the WBS region of chromosome 7.

5. The method of claim 4 wherein the two inversions comprise a first inversion comprising a centromeric breakpoint between the binding sites of probes RP11-815K3 and CTA-208H19 on chromosome 7 and a telomeric breakpoint between the binding sites of probes RP5-1186P10 and RP11-229D13 on chromosome 7 and a second inversion comprising a centromeric breakpoint between the binding sites of probes CTA-208H19 and RP-1186P10 on chromosome 7 and a telomeric breakpoint between the binding sites of probes CTB-139P11 and RP11-275g11 on chromosome 7.

6. The method of claim 1 wherein the biological sample is a nucleic acid sample and the assay is selected from the group consisting of probe hybridization, direct sequencing, restriction enzyme fragment analysis and fragment electrophoretic mobility.

7. The method of claim 6 wherein the nucleic acid sample is a DNA sample.

8. The method of claim 7 wherein the DNA sample is a genomic DNA sample and the assay comprises the steps of:
(a) amplifying a target portion of the nucleotide sequence of the genomic DNA;
(b) obtaining the nucleotide sequence of said amplified target portion; and
(c) determining the presence or absence of an inversion in said target portion nucleotide sequence.

9. The method of claim 8 wherein step (c) comprises determining the presence or absence of an inversion comprising a centromeric breakpoint between the binding sites of probes RP11-815K3 and CTA-208H19 on chromosome 7 and a telomeric breakpoint between the binding sites of probes RP5-1186P10 and RP11-229D13 on chromosome 7 or an inversion comprising a centromeric breakpoint between the binding sites of probes CTA-208H19 and RP-1186P10 on chromosome 7 and a telomeric breakpoint between the binding sites of probes CTB-139P11 and RP11-275g11 on chromosome 7.

10. The method of claim 7 wherein the DNA sample is a genomic DNA sample and the assay comprises:
reacting the genomic DNA with a restriction enzyme to produce restriction fragments; and
analyzing the restriction fragments for at least one fragment indicative of an inversion.

11. The method of claim 10 wherein the restriction enzyme is Not1 and the fragment is a 500 to 600 kb fragment.

12. The method of claim 1 wherein the sample is a cell sample and the assay comprises:
contacting the cells with at least three detectable probes capable of hybridizing to the WBS region of chromosome 7; and
determining the sequential order in which the probes are arrayed after hybridization to the WBS region;
wherein a reversal of the sequential order of any two of the probes relative to the order observed when the probes are hybridized to normal control DNA indicates an inversion of the WBS region of chromosome 7.

13. The method of claim 12 wherein the cells are contacted with two detectable probes which hybridize within the WBS region and one detectable probe which hybridizes telomeric to the WBS region.

14. The method of claim 13 wherein the cells are contacted with probes 208H19, 1186P10 and 139P11.

15. The method of claim 12 wherein the cells are contacted with two detectable probes which hybridize within the WBS region and one detectable probe which hybridizes centromeric to the WBS region.

16. The method of claim 15 wherein the cells are contacted with probes 815K3, 208H19 and 1186P10.


SEQUENCE LISTING (1 sequence)

SEQ ID NO: 1 - DNA, 4 bp, Homo sapiens
  misc_feature (1)..(4): polymorphic repeat marker
taga